(19) United States
(12) Patent Application Publication
(10) Pub. No.: US 2004/0248817 A1
(43) Pub. Date: Dec. 9, 2004

(54) Gamma crystalline form of perindopril tert-butylamine salt

(76) Inventors: Bruno Pfeiffer, Saint Leu La Foret (FR); Yves-Michel Ginot, Orleans (FR); Gerard Coquerel, Boos (FR); Stephane Beilles, Dijon (FR)

Correspondence Address: The Firm of Hueschen and Sage, 500 Columbia Plaza, 350 East Michigan Avenue, Kalamazoo, MI, 49007, US

(21) Appl. No.: 10/811,727

(22) Filed: Mar. 29, 2004

Related U.S. Application Data
(63) Continuation of application No. 10/312,903, filed on Dec. 31, 2002, now abandoned.

(30) Foreign Application Priority Data
Jul. 6, 2000 (FR) 00.08791
Jul. 6, 2001 (WO) PCT/FR01/02169

(51) Int. Cl. A61K 38/04; C07K 5/04; C07D 209/42

(52) U.S. Cl. 514/19; 548/492

(57) ABSTRACT

A γ crystalline form of the compound of formula (I):

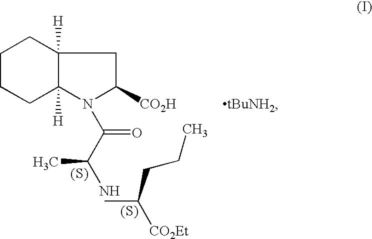

characterized by its powder X-ray diffraction data.

Medicinal products containing the same which are useful as inhibitors of angiotensin I converting enzyme.

The present invention relates to a new γ crystalline form of perindopril tert-butylamine salt of formula (I):

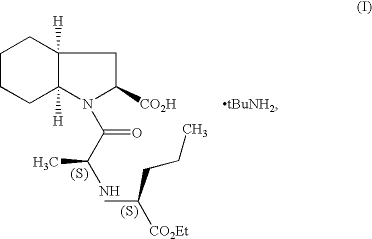

to a process for its preparation and to pharmaceutical compositions containing it.

Perindopril and its pharmaceutically acceptable salts, and more especially its tert-butylamine salt, have valuable pharmacological properties.

Their principal property is that of inhibiting angiotensin I converting enzyme (or kininase II), which prevents, on the one hand, conversion of the decapeptide angiotensin I to the octapeptide angiotensin II (a vasoconstrictor) and, on the other hand, degradation of bradykinin (a vasodilator) to an inactive peptide.

Those two actions contribute to the beneficial effects of perindopril in cardiovascular diseases, more especially in arterial hypertension and heart failure.

Perindopril, its preparation and its use in therapeutics have been described in European Patent specification EP 0 049 658.

In view of the pharmaceutical value of this compound, it has been of prime importance to obtain it with excellent purity. It has also been important to be able to synthesise it by means of a process that can readily be converted to the industrial scale, especially in a form that allows rapid filtration and drying. Finally, that form had to be perfectly reproducible, easily formulated and sufficiently stable to allow its storage for long periods without particular requirements for temperature, light, humidity or oxygen level.

The patent specification EP 0 308 341 describes an industrial synthesis process for perindopril. However, that document does not specify the conditions for obtaining perindopril in a form that exhibits those characteristics in a reproducible manner.

The Applicant has now found that a particular salt of perindopril, the tert-butylamine salt, can be obtained in a well defined, perfectly reproducible crystalline form that especially exhibits valuable characteristics for formulation.

More specifically, the present invention relates to the γ crystalline form of the compound of formula (I), characterised by the following powder X-ray diffraction diagram, measured using a Siemens D5005 diffractometer (copper anticathode) and expressed in terms of inter-planar distance d, Bragg's angle 2 theta, intensity and relative intensity (expressed as a percentage of the most intense ray):

| Angle 2 theta | Inter-planar | | Relative intensity |
|---|---|---|---|
| (°) | distance d (Å) | Intensity | (%) |
| 6.298 | 14.02 | 630 | 39.8 |
| 7.480 | 11.81 | 380 | 24 |
| 8.700 | 10.16 | 1584 | 100 |
| 9.276 | 9.53 | 318 | 20.1 |
| 10.564 | 8.37 | 526 | 33.2 |
| 11.801 | 7.49 | 54 | 3.4 |
| 12.699 | 6.96 | 86 | 5.4 |
| 13.661 | 6.48 | 178 | 11.2 |
| 14.095 | 6.28 | 163 | 10.3 |
| 14.332 | 6.17 | 290 | 18.3 |
| 14.961 | 5.92 | 161 | 10.2 |
| 15.793 | 5.61 | 128 | 8.1 |
| 16.212 | 5.46 | 179 | 11.3 |
| 16.945 | 5.23 | 80 | 5.1 |
| 17.291 | 5.12 | 92 | 5.8 |
| 17.825 | 4.97 | 420 | 26.5 |
| 18.100 | 4.90 | 159 | 10 |
| 18.715 | 4.74 | 89 | 5.6 |
| 19.017 | 4.66 | 118 | 7.4 |
| 19.362 | 4.58 | 134 | 8.5 |
| 19.837 | 4.47 | 133 | 8.4 |
| 20.609 | 4.31 | 95 | 6 |
| 21.232 | 4.18 | 257 | 16.2 |
| 21.499 | 4.13 | 229 | 14.5 |
| 21.840 | 4.07 | 127 | 8 |
| 22.129 | 4.01 | 191 | 12.1 |
| 22.639 | 3.92 | 137 | 8.6 |
| 23.000 | 3.86 | 88 | 5.6 |
| 23.798 | 3.74 | 147 | 9.3 |
| 24.170 | 3.68 | 70 | 4.4 |
| 25.066 | 3.55 | 167 | 10.5 |
| 25.394 | 3.50 | 165 | 10.4 |
| 26.034 | 3.42 | 84 | 5.3 |
| 26.586 | 3.35 | 75 | 4.7 |
| 27.541 | 3.24 | 74 | 4.7 |
| 28.330 | 3.15 | 85 | 5.4 |
| 29.589 | 3.02 | 96 | 6.1 |

The invention relates also to a process for the preparation of the γ crystalline form of the compound of formula (I), which process is characterised in that:

either, according to a first embodiment, a solution of perindopril tert-butylamine salt in chloroform is heated at reflux, the solution is then rapidly cooled to 0° C. and, after stirring, the solid obtained is collected by filtration,

or, according to a second embodiment, a solution of perindopril tert-butylamine salt in ethyl acetate is heated at reflux, the solution is rapidly cooled to between 0 and 5° C. and the solid thereby obtained is then collected by filtration. The solid is suspended in chloroform, the suspension is stirred at ambient temperature for from 5 to 10 days, and the solid is then collected by filtration.

In the crystallisation process according to the invention it is possible to use the compound of formula (I) obtained by any process. Advantageously, the compound of formula (I) obtained by the preparation process described in patent specification EP 0 308 341 is used.

In the first embodiment of the process according to the invention, the concentration of the compound of formula (I) in the chloroform is preferably from 150 to 300 g/litre.

In the second embodiment of the process according to the invention, the concentration of the compound of formula (I) in the ethyl acetate is preferably from 70 to 90 g/litre. The concentration, in chloroform, of the solid obtained is preferably from 100 to 150 g/litre.

The invention relates also to pharmaceutical compositions comprising as active ingredient the γ crystalline form of the compound of formula (I) together with one or more appropriate, inert, non-toxic excipients. Among the pharmaceutical compositions according to the invention, there may be mentioned more especially those that are suitable for oral, parenteral (intravenous or subcutaneous) or nasal administration, tablets or dragées, sublingual tablets, gelatin capsules, lozenges, suppositories, creams, ointments, dermal gels, injectable preparations, drinkable suspensions etc.

The useful dosage can be varied according to the nature and severity of the disorder, the administration route and the age and weight of the patient. It varies from 1 to 500 mg per day in one or more administrations.

The pharmaceutical compositions according to the invention may also comprise a diuretic such as indapamide.

The following Examples illustrate the invention but do not limit it in any way.

The powder X-ray diffraction spectrum was measured under the following experimental conditions:

Siemens D5005 diffractometer, scintillation detector,

copper anticathode (γ=1.5405 Å), voltage 40 kV, intensity 40 mA,

mounting θ-θ

measurement range: 5° to 30°,

increment between each measurement: 0.02°,

measurement time per step: 2 s,

variable slits: v6,

filter Kβ(Ni),

no internal reference,

zeroing procedure with the Siemens slits,

experimental data processed using EVA software (version 5.0).

### EXAMPLE 1

### γ Crystalline Form of Perindopril Tert-Butylamine Salt

100 g of perindopril tert-butylamine salt obtained according to the process described in patent specification EP 0 308 341 are dissolved in 500 ml of chloroform heated at reflux. The solution is then cooled to 0° C. and stirred overnight at that temperature. The solid obtained is collected by filtration.

Powder X-ray Diffraction Diagram:

The powder X-ray diffraction profile (diffraction angles) of the γ form of perindopril tert-butylamine salt is given by the significant rays collated in the following table together with the intensity and relative intensity (expressed as a percentage of the most intense ray)

| Angle 2 theta | Inter-planar | | Relative intensity |
|---|---|---|---|
| (°) | distance d (Å) | Intensity | (%) |
| 6.298 | 14.02 | 630 | 39.8 |
| 7.480 | 11.81 | 380 | 24 |
| 8.700 | 10.16 | 1584 | 100 |
| 9.276 | 9.53 | 318 | 20.1 |
| 10.564 | 8.37 | 526 | 33.2 |
| 11.801 | 7.49 | 54 | 3.4 |
| 12.699 | 6.96 | 86 | 5.4 |
| 13.661 | 6.48 | 178 | 11.2 |
| 14.095 | 6.28 | 163 | 10.3 |
| 14.332 | 6.17 | 290 | 18.3 |
| 14.961 | 5.92 | 161 | 10.2 |
| 15.793 | 5.61 | 128 | 8.1 |
| 16.212 | 5.46 | 179 | 11.3 |
| 16.945 | 5.23 | 80 | 5.1 |
| 17.291 | 5.12 | 92 | 5.8 |
| 17.825 | 4.97 | 420 | 26.5 |
| 18.100 | 4.90 | 159 | 10 |
| 18.715 | 4.74 | 89 | 5.6 |
| 19.017 | 4.66 | 118 | 7.4 |
| 19.362 | 4.58 | 134 | 8.5 |
| 19.837 | 4.47 | 133 | 8.4 |
| 20.609 | 4.31 | 95 | 6 |
| 21.232 | 4.18 | 257 | 16.2 |
| 21.499 | 4.13 | 229 | 14.5 |
| 21.840 | 4.07 | 127 | 8 |
| 22.129 | 4.01 | 191 | 12.1 |
| 22.639 | 3.92 | 137 | 8.6 |
| 23.000 | 3.86 | 88 | 5.6 |
| 23.798 | 3.74 | 147 | 9.3 |
| 24.170 | 3.68 | 70 | 4.4 |
| 25.066 | 3.55 | 167 | 10.5 |
| 25.394 | 3.50 | 165 | 10.4 |
| 26.034 | 3.42 | 84 | 5.3 |
| 26.586 | 3.35 | 75 | 4.7 |
| 27.541 | 3.24 | 74 | 4.7 |
| 28.330 | 3.15 | 85 | 5.4 |
| 29.589 | 3.02 | 96 | 6.1 |

### EXAMPLE 2

### γ Crystalline Form of Perindopril Tert-Butylamine Salt

125 g of perindopril tert-butylamine salt obtained according to the process described in patent specification EP 0 308 341 are dissolved in 1.5 litres of ethyl acetate heated at reflux.

The temperature of the solution is then rapidly brought to between 0 and 5° C.

The solid obtained is then collected by filtration and is then suspended in 750 g of chloroform. The suspension is stirred at ambient temperature for from 5 to 10 days and the solid is then collected by filtration.

### EXAMPLE 3

### Pharmaceutical Composition

Preparation formula for 1000 tablets each containing 4 mg of active ingredient:

Compound of Example 1 . . . 4 g

Hydroxypropylcellulose . . . 2 g

Wheat starch . . . 10 g

Lactose . . . 100 g

Magnesium stearate . . . 3 g

Talc . . . 3 g

1. γ crystalline form of the compound of formula (I): 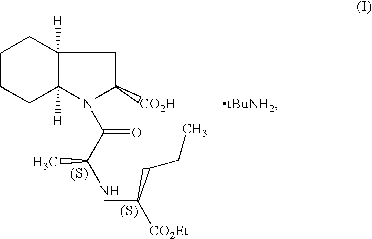
characterised by the following powder X-ray diffraction diagram, measured using a diffractometer (copper anticathode) and expressed in terms of inter-planar distance d, Bragg's angle 2 theta, intensity and relative intensity (expressed as a percentage with respect to the most intense ray):

| Angle 2 theta | Inter-planar | | Relative intensity |
|---|---|---|---|
| (°) | distance d (Å) | Intensity | (%) |
| 6.298 | 14.02 | 630 | 39.8 |
| 7.480 | 11.81 | 380 | 24 |
| 8.700 | 10.16 | 1584 | 100 |
| 9.276 | 9.53 | 318 | 20.1 |
| 10.564 | 8.37 | 526 | 33.2 |
| 11.801 | 7.49 | 54 | 3.4 |
| 12.699 | 6.96 | 86 | 5.4 |
| 13.661 | 6.48 | 178 | 11.2 |
| 14.095 | 6.28 | 163 | 10.3 |
| 14.332 | 6.17 | 290 | 18.3 |
| 14.961 | 5.92 | 161 | 10.2 |
| 15.793 | 5.61 | 128 | 8.1 |
| 16.212 | 5.46 | 179 | 11.3 |
| 16.945 | 5.23 | 80 | 5.1 |
| 17.291 | 5.12 | 92 | 5.8 |
| 17.825 | 4.97 | 420 | 26.5 |
| 18.100 | 4.90 | 159 | 10 |
| 18.715 | 4.74 | 89 | 5.6 |
| 19.017 | 4.66 | 118 | 7.4 |
| 19.362 | 4.58 | 134 | 8.5 |
| 19.837 | 4.47 | 133 | 8.4 |
| 20.609 | 4.31 | 95 | 6 |
| 21.232 | 4.18 | 257 | 16.2 |
| 21.499 | 4.13 | 229 | 14.5 |
| 21.840 | 4.07 | 127 | 8 |
| 22.129 | 4.01 | 191 | 12.1 |
| 22.639 | 3.92 | 137 | 8.6 |
| 23.000 | 3.86 | 88 | 5.6 |
| 23.798 | 3.74 | 147 | 9.3 |
| 24.170 | 3.68 | 70 | 4.4 |
| 25.066 | 3.55 | 167 | 10.5 |
| 25.394 | 3.50 | 165 | 10.4 |
| 26.034 | 3.42 | 84 | 5.3 |
| 26.586 | 3.35 | 75 | 4.7 |
| 27.541 | 3.24 | 74 | 4.7 |
| 28.330 | 3.15 | 85 | 5.4 |
| 29.589 | 3.02 | 96 | 6.1 |

2. Process for the preparation of the γ crystalline form of the compound of formula (I) according to claim 1, characterised in that a solution of perindopril tert-butylamine salt in chloroform is heated at reflux, the solution is then cooled to 0° C. and the solid obtained is collected by filtration.

3. Process for the preparation of the γ crystalline form of the compound of formula (I) according to claim 1, characterised in that a solution of perindopril tert-butylamine salt in ethyl acetate is heated at reflux, the solution is rapidly cooled, the solid thereby obtained is then collected by filtration, it is suspended in chloroform, the suspension is stirred at ambient temperature for from 5 to 10 days, and the solid is then collected by filtration.

4. Process according to either claim 2 or claim 3, characterised in that the compound of formula (I) obtained by the preparation process described in patent specification EP 0 308 341 is used.

5. Process according to claim 2, characterised in that the concentration of the compound of formula (I) in the chloroform is from 150 to 300 g/litre.

6. Process according to claim 3, characterised in that the concentration of the compound of formula (I) in the ethyl acetate is from 70 to 90 g/litre.

7. Pharmaceutical composition comprising as active ingredient the compound according to claim 1, in combination with one or more pharmaceutically acceptable, inert, non-toxic carriers.

8. Pharmaceutical composition according to claim 7 for use in the manufacture of medicaments for use as inhibitors of angiotensin I converting enzyme.

9. Pharmaceutical composition according to claim 8 for use in the manufacture of medicaments for use in the treatment of cardiovascular diseases.

10. Pharmaceutical composition according to any one of claims 7 to 9, characterised in that it also comprises a diuretic.

11. Pharmaceutical composition according to claim 10, characterised in that the diuretic is indapamide.
